(19) United States
(12) Patent Application Publication
(10) Pub. No.: US 2002/0001797 A1
(43) Pub. Date: Jan. 3, 2002

(54) Method for detecting endocrine disrupting action of a test substance

(76) Inventors: Mitsuko Ishihara, Tokyo (JP); Eiichi Akahoshi, Kawasaki-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/892,485

(22) Filed: Jun. 28, 2001

(30) Foreign Application Priority Data
Jun. 30, 2000 (JP) 2000-198479
Dec. 28, 2000 (JP) 2000-401633

(51) Int. Cl. C12Q 1/00; C12Q 1/68

(52) U.S. Cl. 435/4; 435/6

(57) ABSTRACT

The present invention relates to a method of detecting an endocrine disrupting action of a test substance and also relates to a polynucleotide and a complementary nucleotide specifically expressed in a cell by the endocrine disrupting action, and a DNA chip having either the polynucleotide or the complementary polynucleotide. The present invention further relates to an abnormally-modified protein biosynthesized in a cell specifically by the endocrine disrupting action and an antibody of the abnormally modified protein and a DNA chip provided with the antibody.

### CROSS-REFERENCE TO RELATED APPLICATIONS

This application is based upon and claims the benefit of priority from the prior Japanese Patent Applications No. 2000-198479, filed Jun. 30, 2000; and No. 2000-401633, filed Dec. 28, 2000, the entire contents of both of which are incorporated herein by reference.

### BACKGROUND OF THE INVENTION

The present invention relates to a method of detecting an endocrine disrupting action of a test substance. The present invention also relates to a specific polynucleotide to the endocrine disrupting action, and a polynucleotide having a complementary sequence to that of the specific polynucleotide, and a DNA chip having at least either one of these polynucleotides. Furthermore, the present invention relates to an abnormally-modified protein and its antibody, and a protein chip having the antibody.

Recently, it has been found that the chemical substances present in the environment exhibit both hormone-like and anti-hormonal actions. Due to such actions, these chemical substances are generally called “endocrine disrupting substances”.

It has gradually become clear that the endocrine disrupting substances cause abnormalities not only in the reproductive system but also in the nervous system and the immune system due to their endocrine disrupting actions. At present, at least 70 types of chemical substances, most notably, dioxin, are suspected as endocrine disrupting substances. It is estimated that endocrine disrupting substances will increase in number from now on. Under these circumstances, the ability to detect the endocrine disrupting substances will become more and more important.

Conventional methods for detecting the endocrine disrupting actions include an E-SCREEN method (disclosed in Soto A. M. et al., Environ. Health Perspect, 103 pp113-122 (1995), a Two-hybrid assay (Nishikawa J. et al. Toxcol. Appl. Pharmacol. 154 pp76-83 (1999)), and so on. However, these conventional methods are directed to detecting a hormone-like action, in particular, an estrogen-like action of a test substance. Therefore, an endocrine disrupting action other than an estrogen-like action cannot be detected by conventional methods.

### BRIEF SUMMARY OF THE INVENTION

An object of the present invention is to provide a method of specifically detecting an endocrine disrupting action of a test substance. More specifically, the object of the present invention is to provide a method of detecting endocrine disrupting action of a test substance, which is capable of detecting not only an estrogen-like action but also other hormonal actions and an anti-hormonal action.

According to an aspect of the present invention, there is provided a method of detecting an endocrine disrupting action of a test substance comprising:

(1) culturing a cell having a sensitivity to an endocrine hormone in a first culture system in which the endocrine hormone and the test substance are present; and

(2) determining the presence or absence of an endocrine disrupting action of the test substance by comparing a first gene expression pattern obtained from the cell of the first culture system with a second gene expression pattern expressed by a cell having a sensitivity to the endocrine hormone in the presence of the endocrine hormone, thereby detecting a gene specific to the first gene expression pattern.

According to another aspect of the present invention, there is provided a method of detecting an endocrine disrupting action of a test substance, comprising:

(1) (a) culturing a cell having a sensitivity to an endocrine hormone in a first culture system in which an endocrine hormone and the test substance are present; and

(b) culturing the cell a having a sensitivity to the endocrine hormone in a third culture system in which the endocrine hormone is absent and the test substance is present; and

(2) determining the presence or absence of an endocrine disrupting action of the test substance by obtaining a first gene expression pattern obtained from the cell in the first culture system and a third gene expression pattern obtained from the cell in the third culture system, comparing the first gene expression pattern with the third gene expression pattern and a second expression pattern expressed by a cell having a sensitivity to the endocrine hormone in the presence of the endocrine hormone, thereby detecting a gene specific to the first gene expression pattern.

According to still another aspect of the present invention, there is provided a method of detecting an endocrine disrupting action of a test substance, comprising:

(1) (a) culturing a cell having a sensitivity to an endocrine hormone in a first culture system in which the endocrine hormone and the test substance are present; and

(b) culturing the cell a having a sensitivity to the endocrine hormone in a third culture system in which the endocrine hormone is absent and the test substance is present; and

(2) determining the presence or absence of an endocrine disrupting action of the test substance by comparing a first gene expression pattern obtained from the cell of the first culture system with a third gene expression pattern obtained from the cell in the third culture system, thereby detecting a gene specific to the first gene expression pattern.

According to a further aspect of the present invention, there is provided a method of detecting an endocrine disrupting action of a test substance, comprising the steps of:

(1) culturing a cell having a sensitivity to an endocrine hormone in a first culture system in which the endocrine hormone and the test substance are present; and

(2) determining the presence or absence of an endocrine disrupting action of the test substance by comparing a first glycoprotein pattern obtained from the cell of the first culture system with a second glycoprotein pattern expressed by a cell having a sensitivity to the endocrine hormone in the presence of the endocrine hormone, thereby detecting a glycoprotein specific to the first glycoprotein pattern.

According to an additional aspect of the present invention, there is provided a method of detecting an endocrine disrupting action of a test substance, comprising the steps of:

(1) (a) culturing a cell having a sensitivity to an endocrine hormone in a first culture system in which the endocrine hormone and the test substance are present; and

(b) culturing a cell having a sensitivity to the endocrine hormone in a third culture system in which the endocrine hormone are absent and the test substance are present; and

(2) determining the presence or absence of endocrine disrupting action of the test substance by obtaining a first glycoprotein pattern obtained from the cell of the first culture system and a third glycoprotein pattern obtained form the cell of the third culture system, and

comparing the first glycoprotein pattern with the third glycoprotein pattern and a second glycoprotein pattern obtained from a cell having a sensitivity to the endocrine hormone in the presence of the endocrine hormone, thereby detecting a glycoprotein specific to the first glycoprotein pattern.

According to still a further object of the present invention, there is provided a method of detecting an endocrine disrupting action of a test substance, comprising the steps of:

(1) (a) culturing a cell having a sensitivity to an endocrine hormone in a first culture system in which the endocrine hormone and the test substance are present; and

(b) culturing a cell having a sensitivity to the endocrine hormone in a third culture system in which the endocrine hormone is absent and the test substance is present; and

(2) determining the presence or absence of endocrine disrupting action of the test substance by

comparing the first glycoprotein pattern obtained from the cell of the first culture system with the third glycoprotein pattern obtained from the cell of the third culture system, thereby detecting a glycoprotein specific to the first glycoprotein pattern.

### DESCRIPTION OF THE SEVERAL VIEWS OF THE DRAWING

FIG. 1 is a view showing the concept of a method for detecting endocrine disrupting properties of a test substance according to an embodiment of the present invention;

FIG. 2 is a view showing the concept of a conventional method;

FIG. 3 is a scheme showing a method of obtaining a transcriptional product according to an embodiment of the present invention;

FIG. 4A is a scheme showing a method of obtaining a transcriptional product according to an embodiment of the present invention;

FIG. 4B is a view showing bands of PCR flagments on a gel according to an embodiment of the present invention;

FIG. 5 is a scheme showing a biosynthetic process of a glycoprotein; and

FIG. 6 is a scheme showing a method of detecting endocrine disrupting actions of a test substance according to an embodiment of the present invention.

### DETAILED DESCRIPTION OF THE INVENTION

The term “endocrine disrupting substance”, which is the target of the detection of the present invention, refers to an exogenous chemical substance. The exogenous chemical substance is one which inhibits or accelerates biological processes such as synthesis, storage, secretion, internal transportation, receptor binding, hormonal action and excretion of various endogenous hormones regulating homeostasis, reproduction, development, and behavior. The exogenous chemical substance is also known as an exogenous endocrine disrupting substance, an endocrine disrupting substance, an endocrine disordering substance, or an environmental hormone. The terms “exogenous endocrine disrupting substance”, “endocrine disrupting substance”, and “endocrine disordering substance” can be interchangeably used in this text.

The term “endocrine disrupting action” refers to the action of disrupting the function of an endocrine hormone, thereby destroying natural cellular homeostasis maintained by an endocrine hormone. More specifically, the endocrine disrupting substance binds to a hormone receptor present in a nucleus of a cell, thereby disrupting a spontaneous action induced by binding a proper endocrine hormone to the receptor.

The present invention has been attained by focusing on the mechanism for expressing the endocrine disrupting action. According to an embodiment of the present invention, it is possible to accurately detect the action of endocrine disrupting substances. The endocrine disrupting substances may chronically affect various functions in a biological reproduction system, nervous system, and immune system, even if present in extremely small amounts in the order of ppb to ppt.

According to an embodiment of the present invention, cells used for detecting the action of an endocrine disrupting substance are those having a sensitivity to an endocrine hormone.

The term “cell having a sensitivity to an endocrine hormone” used herein refers to one whose functions are expressed under the control of the endocrine hormone if the hormone is present in its culture system, whereas they are not expressed if the hormone is not present in the culture system. In other words, when the endocrine hormone is present, the function-expressing mechanism controlled by the endocrine hormone operates it is assumed, whereas the function-expressing mechanism does not operate when the endocrine hormone is not present. Also it is assumed that the cell has two control phases regulated by different mechanisms. More specifically, the endocrine hormone acts as a switch for controlling ON and OFF (the initiation and termination) of the expression mechanism in the cell.

Unlike the cells of the present invention, germ cells are used in conventional methods for detecting the action of endocrine disrupting substances. The germ cells are affected by endocrine hormone-like substances regardless of the presence or absence of the endocrine hormone in a culture system. More specifically, the expression of the cellular function is controlled by only one mechanism. For this reason, it is not preferable to employ the conventional cells in the present invention.

The “endocrine hormone” used herein refers to an endogenous hormone secreted from an endocrine gland and received by its receptor present in the nucleus. Examples of an endocrine hormone include steroid hormones including female hormones such as estrogen, estradiol, progesterone; male hormones such as androgen, testosterone and androsterone; adrenal cortex hormones such as cortisol, aldosterone, corticosterone, and cortison; and amino acid derivative hormones including thyroid hormones such as triiodothyronine (in general, simply referred to as “T3”), thyroxine (in general, simply referred to as “T4”), and parathyroid hormones.

Examples of cells having a sensitivity to an endocrine hormone used herein may include germ cells and nerve cells derived from mammals such as humans, swine, bovines, rabbits, mice, and rats. These cells may be normal cells or cancer cells. Alternatively, these cells may be obtained by genetic engineering. However, neither the cells constantly controlled nor those uncontrolled by endocrine hormones are preferably used in the method of the present invention. The cells having sensitivity to endocrine hormones are preferably employed in the present invention.

Examples of the cells preferably employed in the present invention include murine neuroblastoma (Neuro2a), murine uterus carcinoma(MCF7), murine testicular Leydig cell(TM3), cell strains TM4 and 15P-1 derived from testicular Sertoli cells, and murine neuroblastoma (S-20Y).

Whether or not the cell has a sensitivity to an endocrine hormone is confirmed as follows. The sensitivity of Neuro2a is checked by observing a morphological change induced by addition of triiodothyronine. The morphological change is presumably caused by something which projects like a neural spine due to the addition of triiodothyronine. On the other hand, in the case of S-20Y, a cell proliferation rate changes by addition of triiodothyronine. Therefore, it is preferable to employ either the combination of Neuro2a and triiodothyronine or the combination of S-20Y and triiodothyronine since significant changes occur.

Alternatively, it is useful to select an endocrine hormone affected by, susceptible to, or possibly affected by an endocrine disrupting substance and select a cell having a sensitivity to the endocrine hormone in specifically detecting the action of the endocrine disrupting substance.

According to an aspect of the present invention, there is provided a method for detecting the endocrine disrupting action of an endocrine disrupting substance. More specifically, the endocrine disrupting action can be detected based on data obtained from the cells under at least two conditions below.

In a first condition, an endocrine hormone and a test substance are copresent. When the cell having a sensitivity to the endocrine hormone is placed under the first condition, the cell is controlled by the endocrine hormone. Under the first condition, a test substance acts upon the cells. The state observed in this case is more closer to an in-vivo state where the endocrine disrupting substance acts.

In a second condition, an endocrine hormone is present but a test substance is absent. Under the second condition, the cell having a sensitivity to the endocrine hormone is controlled by the endocrine hormone. Since the test substance is absent herein, a single action of the endocrine hormone in the cell can be observed. In other words, the regular action of the endocrine hormone carried out in the cell is observed.

The phenomena caused by the action of a test substance in the cell can be detected by subtracting the phenomena observed in the second condition from those observed in the first condition. The resultant phenomena include a phenomenon derived from the endocrine disrupting action of the test substance and a phenomenon derived from the action of the test substance by itself. The phenomenon derived from the endocrine disrupting action may be, for example, gene expression, protein expression, and protein modification. Furthermore, according to an aspect of the present invention, there is provided a method of specifically detecting only the endocrine disrupting action of an endocrine disrupting substance. To be more specific, the endocrine disrupting action can be specifically detected based on the data obtained from the cells under first and second conditions plus data obtained from the cell under a third condition.

In the third condition, the endocrine hormone is absent, whereas a test substance is present. Since the endocrine hormone is absent, the hormonal control of the cell sensitive to the endocrine hormone is not turned on. Accordingly, the action of the test substance by itself in the cell can be observed. The action of the test substance by itself is acute toxicity, subacute toxicity, or the like, which is caused by the chemical action of the test substance alone.

The phenomena specifically caused by the endocrine disrupting action of the test substance to the cell can be observed by subtracting the phenomena observed in the second and third conditions from the phenomena observed in the first condition. Examples of the resultant phenomena may be gene expression, protein expression, and protein modification. The endocrine disrupting action of the test substance is detected by detecting such a specific phenomenon.

Furthermore, a fourth condition in which both the endocrine hormone and the test substance are absent, may be set. Since the endocrine hormone is absent, the hormonal control of the cell sensitive to the endocrine hormone is not turned on. In addition, since the test substance is not present, the regular action of the cell in the absence of the hormonal control is observed. When the phenomena observed under the fourth condition together with the second and third conditions are subtracted from the phenomena observed in the first condition, the endocrine disrupting action can be more specifically observed.

However, if the difference in the regular action exhibited by the cell sensitive to the endocrine hormone between the cases controlled or uncontrolled by the endocrine hormone, is negligibly small, it is not necessary to subtract the phenomena under the fourth condition. Similarly, if it has been previously known that the action of the test substance by itself is negligibly small, it is not necessary to subtract the phenomena under the third condition.

Now, a method using an expression pattern of a gene as an index and a method using a biosynthetic pattern of a modified protein as an index will be described.

I. Method Using Gene Expression as an Index

The term “gene expression pattern” used herein refers to total or partial information with regard to gene expression such as the type of a gene expressed in a cell under an arbitrary condition, the expression amount of the gene, or whether a specific gene is expressed or not. The range of the gene expression pattern may be arbitrarily selected by a test performer depending upon various conditions.

The term “a gene specific to a first gene expression pattern” used herein refers to a gene specifically expressed by the action of an endocrine disrupting substance, a gene whose expression is suppressed by the action of an endocrine disrupting substance, and a gene whose expression amount and/or transcription amount varies specifically by the action of an endocrine disrupting substance. The term “variation of the expression amount and/or transcription amount” includes all changes-increase and decrease.

The term “polynucleotide” used herein includes a polynucleotide and oligonucleotide, for the sake of convenience. The “polynucleotide” is defined as a substance having at least two nucleosides bonded via a phosphoric ester bond. Examples of the nucleoside include, but not limited to deoxyribonucleoside and ribonucleoside. The term “oligonucleotide” refers to a substance obtained by polymerizing several to several tens of phosphoric esters of nucleosides, that is, nucleotides bonded via a phosphodiester bond. In other words, the term “oligonucleotide” include, but not limited to an oligoribonucleotide and an oligodeoxyribonucleotide.

According to an aspect of the present invention, the detection method comprises roughly two steps (1) and (2). In Step (1), a cell is cultured under desired conditions. In other words, a gene is expressed by culturing a cell in the desired conditions. In Step (2), the expression patterns of the gene in the cell under different conditions of Step (1) are obtained. Based on the expression patterns, a gene affected by the endocrine disrupting action is detected, thereby determining the endocrine disrupting action of a test substance.

According to an aspect of the present invention, a cell is incubated in a culture system under various conditions to obtain first, second, and third gene expression patterns.

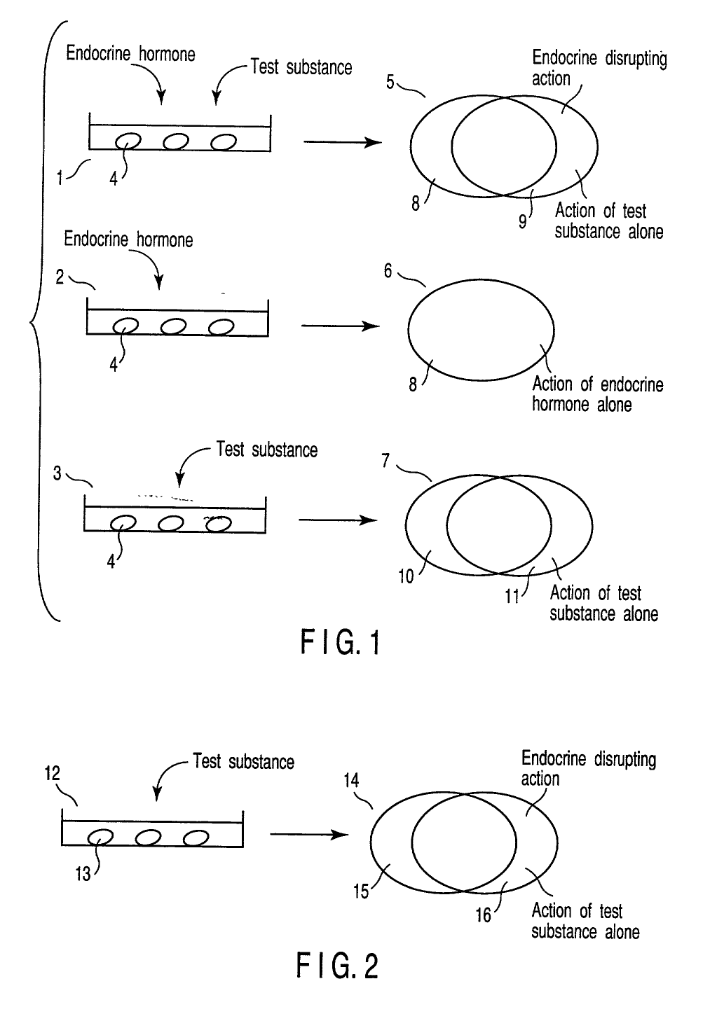

More specifically, in Step (1), a cell is cultured under a first culture system where both an endocrine hormone and a test substance are present. Step (1) will be explained with reference to FIG. 1. In this case, the test substance is regarded as an endocrine disrupting substance, for the sake of convenience.

A cell culture system 1 of FIG. 1 contains both an endocrine hormone and a test substance. When a cell 4 is cultured in the culture system 1, the genes are expressed. The genes are schematically shown as a gene group 5 at the right-hand side of the culture system 1. In culture system 1, the cell 4 expresses a gene group 8 and a gene group 9. The gene group 8 is generally expressed in the presence of an endocrine hormone. A gene group 9 is expressed by addition of a test substance. More specifically. the gene group 9 includes a gene expressed by the endocrine disrupting action of the test substance to the endocrine hormone and a gene expressed by the action of the test substance by itself. Examples of the gene expressed by the action of the test substance alone may include genes associated with a heat shock protein, an apoptosis-associated protein, and a stress protein.

On the other hand, a cell culture system 2 contains the endocrine hormone alone. The genes expressed in this case are schematically shown as the gene group 6 at the right-hand side of the culture system 2. In the culture system 2, the cell 4 generally expresses the gene group 8 in the presence of the endocrine hormone.

Furthermore, a cell culture system 3 is one to which a test substance alone is added in the absence of an endocrine hormone. The genes expressed by culturing a cell in the cell culture system 3 are schematically shown as a gene group 7 at the right-hand side of the culture system 3. In the culture system 3, the cell 4 generally expresses a gene group 10 which is usually expressed and a gene group 11 which is expressed by addition of a test substance. However, unlike the culture system 1, the gene group 11 expressed by addition of the test substance include only the gene expressed by the action of the test substance by itself. Similarly to the case of the culture system 1, examples of the gene expressed by the action of the test substance alone may include genes associated with a heat shock protein, an apoptosis-associated protein, and a stress protein.

As described, Step (1) is carried out for inducing gene expression in three cell culture systems performed under different conditions.

In this step, a fourth culture system may also be prepared as a control culture system. The fourth culture system contains neither a test substance nor an endocrine hormone. In the control system, gene expression generally observed in a normal cell in the absence of the endocrine hormone can be observed.

In Step (2), the gene group 5, the gene group 6 and the gene group 7 expressed in the step (1) are compared, thereby detecting the gene expressed by the endocrine disrupting action of a test substance to the endocrine hormone. As a result, the endocrine disrupting action of the test substance can be determined.

According to the aspect of the present invention, the present invention is characterized by the use of the cell having a sensitivity to an endocrine hormone. In the cell, a different cellular function is expressed depending upon the presence or absence of the endocrine hormone. It is therefore possible to detect the endocrine disrupting action itself, although impossible in conventional techniques.

FIG. 2 shows a method of evaluating an endocrine disrupting action using a germ cell conventionally employed. When a test substance is added to a cell culture system 12 containing a germ cell 13, a gene group 14 is expressed as shown at the right-hand side of the culture system 12 of FIG. 2. In the germ cell 13 used in the conventional method, the expression of a cellular action is constantly controlled by the an endocrine hormone.

Therefore, when the cell culture system 12 is exposed to a test substance, both an endocrine disrupting action and the action of the test substance by itself are observed in the cell despite the presence or absence of an endocrine hormone. Therefore, the endocrine disrupting action of the test substance alone cannot be detected.

As a means for detecting a gene employed in Step (2) of this method, a UV detection means using ethidium bromide may be used. Alternatively, dNTP labeled with a fluorescence or a radioisotope is used when cDNA is synthesized or PCR is performed, and the dNTP was detected by a fluorescent detection method or auto radiography. Moreover, other well-known detection methods may be employed.

In the above, a cell is cultured in the first to third culture systems or the first to fourth culture systems to obtain first to third gene expression patterns or the first to fourth gene expression patterns. However, it is not always necessary to culture the cell in the second and/or fourth culture systems. The gene expression patterns may be obtained from a data bank.

According to another aspect of the present invention, there is provided a polynucleotide which is expressed or varied in transcriptional amount by the endocrine disrupting action of a test substance.

According to still another aspect of the present invention, there is provided a DNA chip for detecting an endocrine disrupting substance. The DNA chip is characterized by comprising a polynucleotide having the gene described above and a polynucleotide having a complementary sequence to that of the gene.

According to the embodiment(s) of the present invention, the presence or absence of the endocrine disrupting action of a test substance can be obtained based on differences in gene expression pattern of the three genes: a gene expressed in a culture system containing a test substance and an endocrine hormone, a gene expressed in a culture system containing the test substance and no endocrine hormone, and a gene expressed in a culture system containing the endocrine hormone and no test substance. The gene which is differently expressed in the three systems may be isolated in the form of a polynucleotide and put into an industrial use.

The substance to be isolated in the aforementioned embodiment may be a gene, transcriptional product, or reverse transcriptional product, more specifically, DNA RNA or cDNA. Alternatively, the substance may include a polynucleotide which is part of the gene and the transcriptional product, as well as a polynucleotide having a complementary sequence to that of the polynucleotide. These substances may be provided by the present invention.

According to an aspect of the present invention, the polynucleotide thus isolated may be used as a marker gene or as a probe. When used as a probe, the polynucleotide is immobilized on a substrate used in a fluorescent-detection type DNA chip and a current detection type DNA chip. The DNA chip and a DNA array fall within the scope of the present invention.

Once a desired polynucleotide is isolated, it is possible to use the polynucleotide as a probe in the DNA chip. Such a DNA chip enables to simply check the endocrine disrupting actions of numerous samples. The DNA chip to be employed in the present invention may be any DNA chip or DNA array generally employed for detecting genes.

Now, an embodiment of the present invention will be explained more specifically. First, a method of isolating the polynucleotide of the present invention will be explained.

1. Method of Isolating a Polynucleotide

According to an embodiment of the present invention, a polynucleotide is isolated by comparing expression patterns of transcriptional products produced in the presence or absence of an endocrine disrupting substance in a cell sensitive to sexual hormones and thyroid hormones, and selecting the polynucleotide exhibiting a change in expression pattern. The expression patterns may be compared by subtraction and electrophoresis.

More specifically, the polynucleotide of the present invention can be isolated in accordance with the following steps (a), (b) and (c).

(a) In-vitro Assay System of an Endocrine Disrupting Substance in a Cultured Cell

A cell, such as murine Neuro2a, having a sensitivity to androgen, estrogen or triiodothyronine is cultured in a medium not containing the hormone. A cell is simultaneously cultured in at least three culture vessels under the same conditions. Thereafter, a hormone(s), for example, triiodothyronine is added to two of the three culture vessels. Furthermore, to one of the two culture vessels with triiodethyronine and the other culture vessel containing no triiodothyronine, either a test substance or an endocrine disrupting substance, for example, TCDD (2378-tetrachlorobenzo p-dioxin), DDT (dichlorodiphenyl trichloroethane), or the like is added. The cells contained in these three vessels are cultured for 24 hours which may be considered sufficient to observe changes in transcription.

Alternatively, a cell, such as murine Neuro2a, having a sensitivity to androgen, estrogen, or triiodothyronine, is cultured in a medium not containing the hormone. The cell is simultaneously cultured in at least two culture vessels under the same conditions. Thereafter, the above-selected hormone(s), for example, triiodothyronine, is added to the two culture vessels. To one of the two culture vessels, a test substance or an endocrine disrupting substance, for example, TCDD (2378-tetrachlorodibenzo p-dioxin) or DDT (Dichlorodiphenyltrichloroethane) is added. Nothing is added to the other vessel. The two culture vessels are incubated for 24 hours which is considered sufficient to observe changes in transcription.

(b) Isolation of a Transcriptional Product

From each of culture cell samples obtained above, mRNA is isolated as follows. The cultured cells are recovered, denatured with guanidineisocyanate, and subjected to density gradient ultracentrifugation using a cesium solution.

(c) Isolation of Polynucleotide Whose Transcription Amount is Varied Specifically by an Endocrine Disrupting Substance

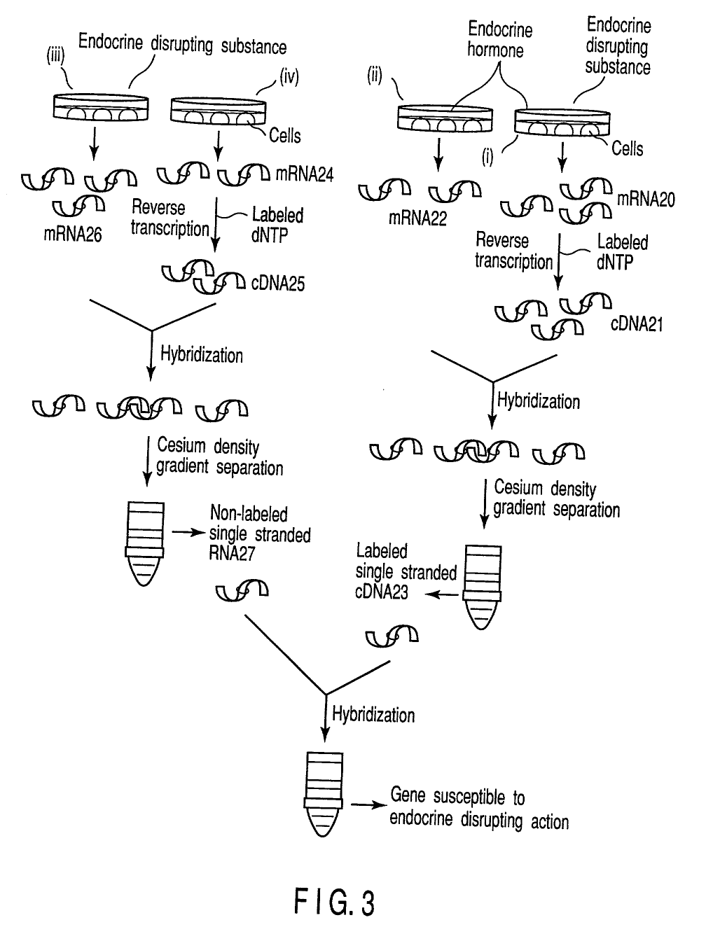

A plurality of types of mRNAs obtained above are compared and then a transcriptional product whose transcriptional amount is specifically varied by addition of an endocrine disrupting substance, is isolated. The isolation is performed by hybridization. This method is called a “subtraction” method which can remove an unnecessary product contained in the cell samples. An example of isolation by the subtraction method is shown in FIG. 3. In this example, four cultivation systems are prepared (see FIG. 3):

(i) a culture system containing an endocrine disrupting substance and an endocrine hormone;

(ii) a culture system containing the endocrine hormone alone;

(iii) a culture system containing the endocrine disrupting substance alone; and

(iv) a control culture system containing neither the endocrine hormone nor the endocrine disrupting substance.

First, mRNA 20 obtained from the cell culture system (i) with the endocrine hormone and the endocrine disrupting substance is subjected to reverse transcription using a reverse transcriptase to obtain cDNA 21. The cDNA 21 is hybridized with mRNA 22 obtained from the cell culture system 22 with no endocrine disrupting substance. The mRNA hybridized is removed by a column or ultracentrifugation.

In the example, ultracentrifugation is used. It is preferable that dNTPs used herein be labeled. In this manner, a transcriptional product cDNA 23 specifically present in the cell cultured in the presence of the endocrine hormone and the endocrine disrupting substance, is obtained (FIG. 3).

Similarly, mRNA 24 obtained from the cell line (iv) is transcribed using a reverse transcriptase to produce cDNA 25. The cDNA 25 is hybridized with mRNA 26 obtained from the cell line (iii). The hybridized mRNA is removed by a column or density gradient ultracentrifugation. In this example, the density gradient ultracentrifugation is employed. It is preferable that dNTPs used herein be labeled. In this method, it is possible to obtain a transcriptional product (designated as RNA 27) of the gene whose expression or transcriptional amount has been changed upon exposure to the endocrine disrupting substance alone (FIG. 3).

The above obtained products, cDNA 23 and RNA 27, are hybridized with each other to thereby obtain cDNA unhybridized, which is a desired transcriptional product specifically related to the action of the endocrine disrupting substance (FIG. 3).

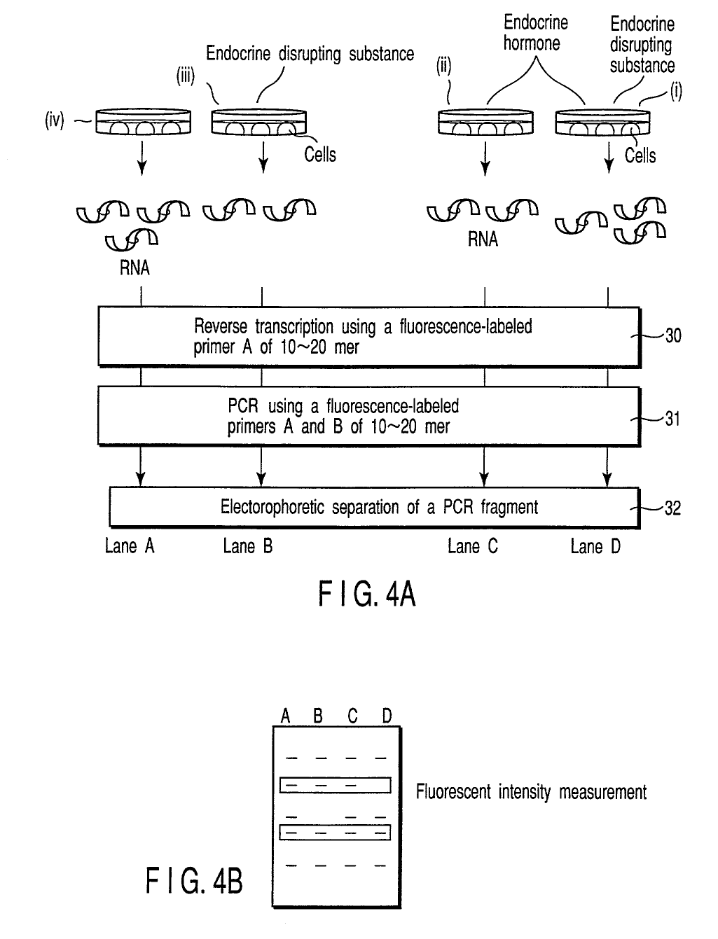

The desired transcriptional product is amplified by a PCR, and then electrophoretically separated. In this way, the desired transcriptional product can be isolated. How to separate the desired transcriptional product by electrophoreses is shown in FIGS. 4A and 4B. In this example, four culture systems are prepared (see FIG. 4A):

(i) a culture system containing an endocrine hormone and an endocrine disrupting substance;

(ii) a culture system containing the endocrine hormone alone;

(iii) a culture system containing the endocrine disrupting substance alone; and

(iv) a control culture system containing neither the endocrine disrupting substance nor the endocrine hormone.

The mRNAs obtained from four cell culture systems are transcribed using primer A having a 10-20 nucleotide sequence and having a label of a fluorescent dye or a radioisotope at an end thereof (see FIG. 4A, reference numeral 30). Thereafter, PCR is performed by using the primer A used in the reverse transcription and a primer B having a 10-20 nucleotide sequence and a label of a fluorescent dye or a radioisotope at an end thereof (FIG. 4A, reference numeral 31). After the PCR, four types of samples are electrophoretically separated in an appropriate gel, e.g., polyacrylamide gel. In this case, the four samples are loaded in lanes next to each other such that the same fragments are electrophoresed at the same migration speed. After the PCR product fragments are migrated sufficiently to separate from each other, the PCR fragments electrophoresed are detected from the gel autoradiographically or by use of a fluoro-imager. Subsequently, the PCR-amplified samples derived from the cell culture system (i), (ii), (iii) and (iv) are compared with each other. The PCR fragment whose amplification amount (determined by the fluorescent intensity) significantly varied, for example, varied twice or more (see FIG. 4B, reference numeral 33 and 34) or a half or less (FIG. 4B, reference numeral 33 and 34), is cut out from the gel. For example, the PCR fragment is recovered by a silica membran. The PCR fragment thus recovered is a polynucleotide of the transcriptional product resulting from the endocrine disrupting action (FIGS. 4A and 4B).

The steps (b) and (c) may be included in the step (2) of this method.

2. DNA Chip

Then, the polynucleotide obtained by the aforementioned method will be used as follows. The polynucleotide can be used as a probe used in dot-blotting, Northern hybridization, or a so-called DNA chip such as a current detection type microarray or a fluorescent microarray.

If a specific polynucleotide or a complementary polynucleotide is detected by a DNA chip containing the polynucleotide or the complementary polynucleotide as a probe, the detection method will be simply and efficiently performed. Examples of the DNA chip includes, but not limited to, a fluorescent-dye DNA chip and a potential-type DNA chip, which differs in detection mechanism.

The DNA chip can be prepared as follows.

(a) Preparation of a Fluorescence-detection Type of DNA Chip

The polynucleotide obtained above or a synthetic oligonucleotide having a partial sequence of the polynucleotide, or a polynucleotide having a complementary sequence to that of the polynucleotide or the oligonucleotide is immobilized to a substrate. As the substrate, any substrate such as a glass substrate or a silicon substrate may be used as long as it is conventionally used. Any immobilization means generally known to those skilled in the art, such as a spotter or a means using a general semiconductor technique, may be used.

(b) Preparation of a Current Detection Type DNA Chip

The polynucleotide of the present invention, that is, the polynucleotide obtained above or an artificially-synthesized oligonucleotide having a partial sequence of the polynucleotide, or the polynucleotide having a complementary sequence to that of the polynucleotide or the oligonucleotide is immobilized to a substrate, for example, an electrode substrate, by means of physical adsorption or chemical adsorption including covalent bonding and ionic bonding. Examples of the current detection type DNA chip may be, but not limited to an automatic DNA detection device registered under U.S. Pat. No. 2,573,443 on Oct. 24, 1996. This patent publication is incorporated herein by reference.

According to the aspect of the present invention, it is possible to easily detect a transcriptional product which specifically varies depending upon the endocrine disrupting action, by use of the gene probe or the DNA chip having the polynucleotide(s).

(c) Method of Detecting Endocrine Disrupting Action by Using a DNA Chip

The endocrine disrupting actions of a large number of test substances are detected without fail by use of the DNA chip of the present invention. To be more specific, first, the polynucleotide resulting from the endocrine disrupting action is detected and isolated by the detection method comprising Step (1) and Step (2). Then, the polynucleotide thus obtained is immobilized to a desired substrate to form a DNA chip.

Any type of cell may be used as a sample to be subjected to the DNA chip. The test can be performed by allowing a test substance to act upon the arbitrarily chosen cell under the control of an endocrine hormone. To explain more specifically, a test substance is act upon a cultured system of the cell. A desired polynucleotide is prepared by extracting, for example, mRNA from the cell-cultured system. The desired polynucleotide is set in a DNA chip and hybridization is performed. With this procedure, the gene susceptible to the endocrine disrupting action can be easily checked in a short time. In addition, even if numerous samples are used, the tests can be performed in a short time. The nucleotide extracted from the culture system and the probe to be immobilized on the DNA chip are preferably designed so as to have complementary sequences to each other. In this case, any known label such as a fluorescence or a radioisotope may be used as a detection label.

II. A Detection Method Using a Modified Protein as an Indicator

According to an aspect of the present invention, there is provided a method for specifically detecting the endocrine disrupting action of a test substance based on a conformational change of a polysaccharide chain caused by the post-translational modification of a protein.

According to an embodiment of the present invention, polysaccharide chains of proteins biosynthesized in the cell (in the step (1) of a method using gene expression as an indicator) are compared to each other and then difference in polysaccharide chain structure of proteins produced under different conditions is detected. Through these steps, the endocrine disrupting action of a test substance can be detected.

The polysaccharide chain is added, by an enzymatic reaction, to a protein translated from a gene. The polysaccharide chain plays an important role in a life action regarding cell adhesion, cell recognition, and construction of biological tissues. Therefore, the conformational change of the polysaccharide chain may have a significant effect upon the life action. To be more specific, in a nerve cell adhesion molecule (NCAM), if polysaccharide chains of a polysialic acid decrease in number, the adhesion of the nerve cells is affected. As a result, the formation of the cerebral cortex layer is damaged (Seki T et al., The Journal of Neuroscience 18, pp 3757-3766 (1998)).

On the other hand, it has been reported that the administration of estrogen to a rat changes the polysaccharide chain structure of progestational hormone (Shylaja M., et al. Proceedings of National Academy of Science, USA 90 (1993)). It is suggested that the endocrine disrupting substance may influence not only upon gene expression but also the post-translational modification of a protein.

The terms “endocrine hormone” and “cell having a sensitivity to the endocrine hormone” herein are the same as defined above.

The term “biologically synthesized glycoprotein” used herein is produced by translating a gene expressed in a cell into a peptide chain, and modifying the peptide chain in various ways. The term “polysaccharide chain” used herein refers to a sugar moiety constituting a part of a glycoprotein.

The term “glycoprotein pattern” provides total or partial information on the types of polysaccharide chains contained in a glycoprotein biosynthesized in a cell under arbitrary conditions or on how largely or how significantly the polysaccharide chain is modified. The range of the pattern exhibiting a polysaccharide chain may be arbitrarily set by a test performer depending upon various conditions.

The term “glycoprotein characteristic in a first glycoprotein pattern” refers to one whose sugar-chain structure is affected somewhere in the polysaccharide chain modification stages by an endocrine disrupting action.

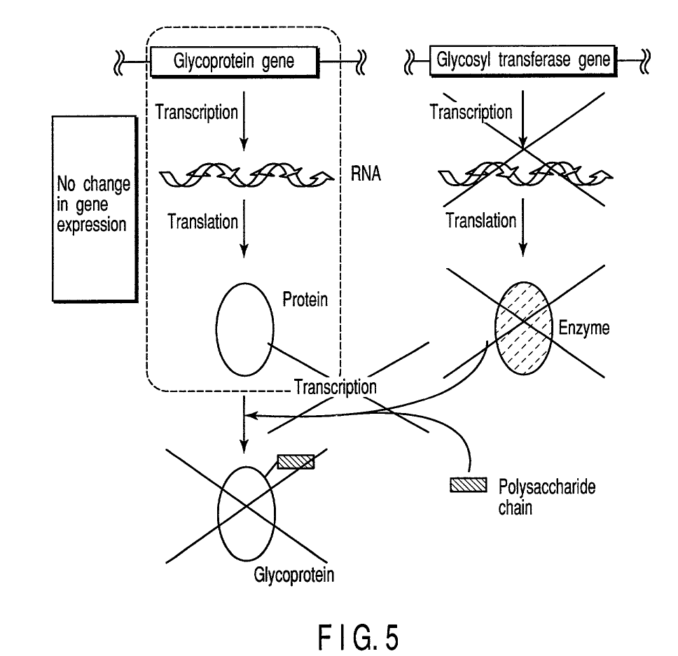

The biosynthetic process of the glycoprotein is shown in FIG. 5. First, RNA is produced by transcription of a glycoprotein gene and translated into a protein. The protein is then modified to accomplish a glycoprotein. Therefore, according to an embodiment of the present invention, the indicator for detecting the endocrine disrupting action is abnormality of the modification process of a protein. Examples of the abnormality include a structural abnormality of a polysaccharide chain caused in the transcriptional stage and translation stage of a glycosyl transferase gene, the expression stage of an enzymatic action and the transfer reaction stage by the transferase.

Now, the embodiments of the present invention will be explained more specifically.

1. Method for Assessing the Presence or Absence of the Endocrine Disrupting Action of a Test Substance

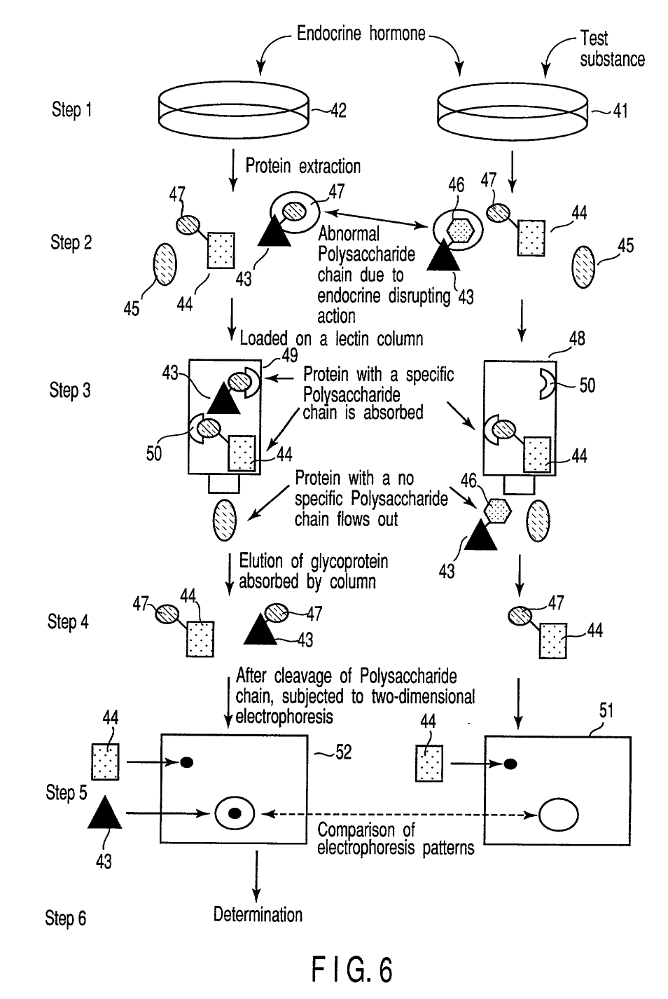

A basic procedure of the present invention will be explained with reference to FIG. 6. In this case, explanation is made provided that a test substance is an endocrine disrupting substance, for the sake of convenience.

First, a culture system 41 and a culture system 42 are prepared. The culture system 41 is prepared to biologically synthesize a protein by incubating a cell having a sensitivity to an endocrine hormone in the presence of the endocrine hormone while exposing the cell to a test substance. The culture system 42 is prepared to biologically synthesize a protein by incubating the cell having a sensitivity to the endocrine hormone in the presence of the endocrine hormone without exposing the cell to the test substance. The cells are incubated in both culture systems for a predetermined time (Step 1).

Note that a system in which the cell is exposed to the test substance in the absence of the endocrine hormone, is required for specifically detecting only the endocrine disrupting action of the endocrine disrupting substance. However, the explanation of the system is omitted herein. The pattern of the glycoproteins biologically synthesized in the cell exposed to a test substance in the absence of the endocrine hormone is also subtracted from the glycoprotein pattern obtained in the culture system 41 in the same manner as in the case of the glycoprotein pattern obtained in the culture system 42. The treatment of this case is performed in substantially the same manner as in the case of the culture systems 41 and 42. The three culture systems are used based on the same principal underlying in the case where a gene is used as an indicator.

As a next step, the biosynthesized protein is extracted (Step 2). Examples of the biosynthesized protein obtained in the culture system 42 include glycoproteins 43 and 44 having a normal polysaccharide chain 47 and a glycoprotein 45 having no polysaccharide chain. The culture system 41 contains a protein 44 having the normal polysaccharide chain 47 (unsusceptible to the effect of a test substance), the protein 45 having no polysaccharide chain in the same as in the culture system 42 and further a protein 44 having a polysaccharide chain 46 affected by the test substance.

As a next step, a solution containing the protein extracted from each of the culture systems is applied to a column in which a substance 50 capable of specifically absorbing a specific polysaccharide chain is immobilized (Step 3). Thereafter, the glycoprotein absorbed to the column is eluted (Step 4). The glycoprotein is recovered and then its polysaccharide chain is cut. The protein with no polysaccharide chain is subjected to electrophoretic separation (Step 5). The electrophoretic patterns of proteins are compared with each other to evaluate whether or not the test substance has the endocrine disrupting action (Step 6).

Steps 1-5 are carried out in almost the same conditions except that a test substance in contained in the culture system 41 and not contained in the culture system 42 in Step 1. This is made to allow comparison between two culture systems.

In Step 3, the glycoproteins 43 and 44 having the normal polysaccharide chain 47 are absorbed to the column 49. In contrast, the glycoprotein 43 having the polysaccharide chain 46 flows out without being absorbed. This is because the polysaccharide chain 46 causes a conformational change by an endocrine disrupting substance and therefore differs from the polysaccharide chain 47 in structure. In addition, the protein 45 with no polysaccharide chain also flows out (Step 3). Therefore, when the separation pattern 51 of the protein derived from the culture system 41 is compared to the separation pattern 52 of the protein derived from the culture system 42, the difference in pattern can be detected (Step 5). Even if the proteins have the same polysaccharide chain, the polysaccharide chain is partly modified in some cases. In this case of the partial modification, difference in expression amount is observed in the electrophoresis separation pattern (Step 5). On the other hand, it may be possible that the transfer of the polysaccharide chain may be accelerated by the endocrine disrupting substance. Also in this case, the difference in the expression amount can be detected. Therefore, if the proteins are compared with each other by using the electrophoretic separation patterns, it is possible to detect the change in polysaccharide chain structure.

The incubation time of step 1 may be arbitrarily set as long as it allows biosynthesis of a protein. The incubation time may be varied depending upon the cell to be used. For example, when a Neuro2a strain is used, the incubation time is preferably set between about 24 hours and about 72 hours.

The term “a substance having a polysaccharide chain binding property” refers to a substance absorbing a polysaccharide chain contained in a glycoprotein. According to an embodiment of the present invention, examples of substance having an acceptable polysaccharide chain binding property include lectin, antibody, ganglioside and Le^{X}. It is preferable to select the substance having a polysaccharide chain binding property depending upon the type of a desired polysaccharide chain.

According to the embodiment of the present invention, examples of the acceptable sugar-chain cleaving enzyme include glycopeptidase F, N-glycanase, and glycopeptidase A. The conditions for cleaving the polysaccharide chain may be appropriately set depending upon the enzyme to be used.

In Step 5, after the polysaccharide chain is cleaved, the resultant protein is electrophoretically separated for comparison. Any separation means may be used as long as it is generally used for separating a protein. For example, the separation may be carried out by electrophoresis, high speed liquid chromatography, or column chromatography. From the analytic point of view, two-dimensional electrophoresis is more preferable.

2. Antibody Against a Polysaccharide Chain Whose Structure is Varied by an Endocrine Disrupting Substance

According to another aspect of the present invention, there is provided an antibody against a polysaccharide chain whose structure is varied upon the influence of an endocrine disrupting substance. According to still another aspect of the present invention, there is a provided a method of directly detecting the polysaccharide chain by use of the antibody, thereby evaluating whether or not a test substance has the endocrine disrupting action.

The following operation is carried out in consideration of the data obtained from the culture system having only a test substance added thereto. Also, the data obtained from a control system containing neither a test substance nor an endocrine hormone, are taken into consideration. Therefore, the culture system containing a test substance alone and the control culture system will not be explained herein.

In this method, first, using a protein obtained based on the electrophoretic separation pattern obtained through the aforementioned steps 1 to 5, a monoclonal antibody is prepared against the polysaccharide chain whose structure is changed specifically depending upon the endocrine disrupting substance. Subsequently, using the monoclonal antibody, a desired polysaccharide chain is detected from the protein group obtained from the culture system 42. In this manner, it is possible to efficiently and simply evaluate the endocrine disrupting actions with respect to numerous endocrine disrupting substances.

The antibody against the polysaccharide chain is prepared by the following steps:

(a) isolating a protein specified by an electrophoretic separation pattern 52 obtained in Step and preparing a polyclonal antibody against the isolated protein,

(b) recovering a desired glycoprotein from the protein group derived from the culture system 41 containing a test substance, by the polyclonal antibody, and

(c) recovering a polysaccharide chain from the glycoprotein recovered, thereby preparing a monoclonal antibody against the polysaccharide chain.

In the step (a), a desired protein is isolated from the electrophoretic separation pattern 52 obtained in the step 5. Then, the polyclonal antibody against the protein is prepared. Subsequently, the protein is allowed to bind to the polyclonal antibody to thereby isolating a desired glycoprotein biosynthesized in a cell. The polyclonal antibody may be prepared by immunizing an animal by using the recovered protein as an immunogen.

The means for recovering the protein specified from the separation pattern may be varied depending upon the separation means used. In the case of gel electrophoresis, a band is cut from a gel and a protein recovered by a dispersion method or an electrophoretic extraction method.

Examples of the animal to be immunized with the protein recovered include a rabbit, goat, horse, and sheep. The immunization is performed by injecting the immunogen into any one of the aforementioned animals. Alternatively, the immunogen may be a synthesized oligopeptide prepared by determining the amino acid sequence of the protein recovered and synthesizing oligopeptide based on the amino acid sequence thus determined.

If the polyclonal antibody obtained in the above is immobilized on a carrier, it is possible to easily isolate a desired glycoprotein from the protein group obtained in the aforementioned step 2 by use of the immobilized polyclonal antibody. From the obtained glycoprotein, an antibody recognizing the polysaccharide chain can be prepared.

The monoclonal antibody is prepared in accordance with the following procedure. For example, murine spleen cells are immunized with the desired glycoprotein obtained in the above as an immunogen. The murine spleen cells are fused with myeloma cells to prepare a hybridoma group. Then, a polysaccharide chain moiety is cut off from a protein previously obtained by use of the polyclonal antibody and the polysaccharide chain moiety is immobilized to an appropriate carrier. Subsequently, a hybridoma producing an antibody capable of specifically binding to the polysaccharide chain moiety is screened by use of the moiety immobilized on the carrier. Based on the hybridoma thus screened, the monoclonal antibody is prepared as described below. First, the hybridoma may be cloned by, for example, a limiting dilution method, a soft agar method, or a fibrinogen method. Then, the hybridoma is injected into a mouse abdominal cavity or the like. Thereafter, monoclonal antibodies produced by the hybridoma are recovered. The antibodies obtained in this manner can recognize a polysaccharide chain which changes the conformation by an endocrine disrupting action. In this text, the monoclonal antibody is exemplified; however, it would be clear to a person skilled in the art to understand that a polyclonal antibody also falls in the scope of the present invention. The polyclonal antibody is also prepared in the same manner as in the preparation of the polyclonal antibody of the protein.

Furthermore, according to an embodiment of the present invention, the polysaccharide chain which has caused a conformational change by an endocrine disrupting action can be directly detected by the use of the monoclonal antibody thus obtained.

To describe more specifically, if the protein, which is extracted from the culture system 41, is reacted with the monoclonal antibody obtained in the above, it is possible to specifically detect the polysaccharide chain changed in conformation due to an endocrine disrupting action. More specifically, the aforementioned reaction is an antigen-antibody reaction by which the monoclonal antibody is bound to the glycoprotein. Subsequently, for example, western blotting, ELISA method, RIA method, or fluorescent antibody method may be carried out to detect a desired glycoprotein.

The monoclonal antibody provided by the present invention specifically binds to a polysaccharide chain susceptible to an endocrine disrupting substance. Accordingly, the monoclonal antibody can be used to detect or recover the polysaccharide chain and/or the glycoprotein. Alternatively, a protein chip described later may be manufactured by immobilizing the monoclonal antibody to a substrate.

The polyclonal antibody provided by the embodiment of the present invention may be used in a protein chip and ELISA method.

3. Protein Chip

In an aspect of the present invention, there is provided a protein chip easily attaining the aforementioned detection. Such a protein chip has an antibody recognizing a desired substance arranged as a probe. If the protein chip is used, it is possible to easily and efficiently detect a test substance. Examples of the protein chip include, but not limited to, a fluorescent detection protein chip (generally referred to as “fluorescent protein chip”) and a current-detection type protein chip (generally, referred to as “potential type protein chip”). The method of manufacturing a protein chip will be described below.

(a) Preparation of the Fluorescence Detection Protein Chip

A monoclonal antibody against a desired polysaccharide chain is previously prepared and immobilized on a substrate. Any substance such as a glass substrate or a silicon substrate may be use as long as it is conventionally used. Any immobilization means such as a spotter, or a means using a general semiconductor technique may be used as long as which it is generally known to an artisan. Furthermore, a label to be used for detection, a fluorescent substance, a radioisotope, or a pigment may be used.

(b) Preparation for the Current-detection Type Protein Chip

A monoclonal antibody against a desired polysaccharide chain is previously prepared and immobilized to a substrate to be used in a general gene-sensor type DNA chip to prepare the current-detection type protein chip.

According to an embodiment of the present invention, it is possible to detect the polysaccharide chain causing a conformational change by an endocrine disrupting action by use of the protein chip provided with the aforementioned antibody.

(c) Method for Detecting an Endocrine Disrupting Action by the Protein Chip

If the protein chip is used in a test, it is not necessary to prepare a plurality of culture systems in step 1 of biosynthesizing a protein. First, a cell is cultured in a system having an endocrine hormone and a test substance. A protein is extracted from the cell culture system and treated to the protein chip. Subsequently, an antigen-antibody reaction is performed. In this manner, it is possible to easily and immediately check numerous samples for detecting a glycoprotein having a polysaccharide chain susceptible to an endocrine disrupting action.

As described above, a method for detecting the endocrine disrupting action of a test substance according to the embodiment of the present invention may be performed by the protein chip provided with an antibody previously identified and prepared.

Now, the present invention will be described in detail with reference to specific examples. However the present invention will not be limited to these examples. Any embodiment may be included if it is not exceeds the gist and the scope of the present invention.

### EXAMPLE 1

In-vitro Assay System for an Endocrine Disrupting Substance

1. Culturing Neuro2a

An anionic exchange resin AG1-X8 and fetal bovine serum were mixed at a ratio of 50 mg resin/mL and incubated at room temperature for 5 hours. After centrifugation at 1000×g for 10 minutes, the resin was removed. A fresh resin was added at a ratio of 50 mg resin/mL to the resultant mixture and subjected to incubation at room temperature for 18 hours. Thereafter, centrifugation was performed at 1000×g for 10 minutes and 30,000×g for 20 minutes to completely remove the resin. The resultant mixture was sterilized by a filter having pours of 0.22 μm diameter to prepare triiodothyronine-removed fetal bovine serum.

The serum (10%) was mixed with DF medium containing Dulbecco's MEM medium and ham F-12 medium at a ratio of 1:1. Murine neuroblastoma, Neuro2a was cultured in the 10% fetal bovine serum containing DF medium at 37° C. in a 5% CO₂. Thereafter, 30 nM triiodothyronine was added to the resultant culture solution and subjected to incubation for a further 5 days.

2. In-vitro Assay for Dioxin

The cells obtained in the incubation of Step 1 were distributed into two vessels. To one of the two culture vessels, 1 nM of TCDD (2378-tetrachlorodibenzo p-dioxin), which is an endocrine disrupting substance, was added. TCDD was not added to the other vessel. After both two vessels were incubated for 24 hours, morphological changes of the cells were observed and the cells were recovered.

### EXAMPLE 2

Obtaining a Polynucleotide Whose Transcription Amount Varies Depending Upon an Endocrine Disrupting Substance (1)

1. Preparation for mRNA

To the cells (5×10⁶) obtained in Example 1, 18 mL of homogenate buffer (containing guanidineisocyanate, SDS, EDTA) was added and then homogenized. After the homogenate was centrifuged at 15° C. at 5000×g for 20 minutes, the supernatant was taken. Subsequently, the supernatant was reciprocally moved 10 times through the injection needle of 18G connected to a syringe. The resultant solution was overlaid on the cesium solution (containing CsTFA and EDTA) of the same amount and subjected to ultracentrifugation at 3000×g for 20 hours. After the ultracentrifugation, a centrifuge tube was taken out and the supernatant was gently sucked up by a pipette. The centrifuge tube was turned upside down on paper towel and allowed to dry. After the drying, the bottom of the centrifuge tube was cut off by a heated razor. The sediment (pellet) was washed with 70% ethanol and further dried. Thereafter, the pellet was suspended in sterile water to give a mRNA solution.

2. Subtraction of mRNA

Of mRNA solutions obtained from the two types of cells in Step 1 above, the mRNA solution obtained from the cell incubated in the culture added with TCDD was subjected to reverse transcription to synthesize cDNA. More specifically, cDNA was synthesized as follows: To the solution containing 1 μg of RNA, a reverse transcriptase buffer, dNTP mix (10 μM for each), an RNAase inhibitor (10 u), a random primer (25 pmol), and M-MLV reverse transcriptase (20 u) were added to make up 20 μL of the total volume. The solution was subjected to reactions performed at 30° C. for 10 minutes, 42° C. for 20 minutes, and 99° C. for 5 minutes, to synthesize cDNA. Subsequently, RNase was added in the cDNA solution to remove RNA. With this procedure, a single-stranded DNA solution was prepared. This solution and the mRNA solution obtained in the step (1) from the cells incubated in the medium without TCDD are mixed to allow a hybridization reaction at 37° C. The hybridization reaction solution was overlaid on the cesium solution of the same amount, and subjected to ultracentrifugation at 30,000×g for 20 hours. A DNA solution containing an unhybridized single chain DNA was recovered to afford a desired polynucleotide solution.

### EXAMPLE 3

Obtaining of Polynucleotide Whose Transcription Amount Varies Depending Upon an Endocrine Disrupting Substance (2).

1. Preparation of mRNA

To the cells (5×10⁶) obtained in Example 1, 18 mL of homogenate buffer (containing guanidineisocyanate, SDS, EDTA) was added. The homogenate was centrifuged at 15° C. at 5000×g for 20 minutes and the supernatant was collected. Furthermore, the supernatant was reciprocally moved 10 times in an injection needle of 18G connected to a syringe. The obtained solution was overlaid on the cesium solution (containing CsTFA and EDTA) of the same amount and subjected to ultracentrifugation at 30,000×g for 20 hours. After the ultracentrifugation, a centrifuge tube was taken out and the supernatant was gently sucked up by a pipette. The centrifuge tube was turned upside down on paper towel and allowed to dry. After the drying, the bottom of the centrifuge tube was cut off by a hot razor. The sediment was washed with 70% ethanol and allowed to dry. After the drying, the sediment was suspended in sterile water to prepare a mRNA solution.

2. Comparison of DNA Fragments by PCR

From the mRNA solutions prepared from two types of cells obtained in Step 1, cDNA was synthesize by rverse transcription. More specifically, cDNA was synthesized as follows. To a solution containing 1 μg of RNA, a reverse transcriptase buffer, dNTPmix (10 μM each), RNAase inhibitor (10 u), a primer (25 pmol) having an arbitrary sequence whose end was labeled with rhodamine, and M-MLV reverse transcriptase (20 u) were added to make up 20 μL of the total volume. The solution was subjected to reactions performed at 30° C. for 10 minutes, 42° C. for 20 minutes, and 99° C. for 5 minutes, to synthesize cDNA. To the solution, further a reverse transcriptase buffer, a primer (10 pmol) having an arbitrary sequence whose end was labeled with rhodamine, another primer (10 pmol) labeled with rhodamine at an end, and Taq polymerase (2.5 u) were added to make up 100 μL of the total volume. PCR cycle of 94° C. for 30 seconds, 55° C. for 30 seconds, 72° C. for one minute was repeated for 30 times. As a result, a solution containing a RT-PCR product obtained by amplifying an arbitrary portion.

The solutions respectively containing two types of RT-PCR products were loaded on lanes adjacent to each other formed in 4% polyacrylamide gel containing 7M urine and electrophoresed in a TBE buffer for 3 hours while supplying a constant power of 40W. The gel plate was set under a fluorescent image analyzer to detect bands of the RT-PCR products. The fluorescent intensities of the bands of the adjacent lanes were compared. The bands whose difference in intensity is 2 times or more or a half or less were excised from the gel. From the gel fragment, a RT-PCR fragment was recovered by use of a silica membran to obtain a solution containing a desired polynucleotide fragment.

### EXAMPLE 4

Preparation of a DNA Chip (1)

A DNA chip having a polynucleotide whose transcription amount varies depending upon an endocrine disrupting substance was prepared as follows. The fluorescence detection type DNA chip was manufactured by immobilizing the polynucleotide (serving as a probe) obtained in Example 2 or 3 on a glass substrate coated with polylysine by a commercially available spotter.

### EXAMPLE 5

Preparation of a DNA Chip (2)

A DNA chip on which a polynucleotide whose transcription amount varies depending upon an endocrine disrupting substance immobilized, was prepared as follows. The polynucleotide obtained in Example 2 or 3 was treated with terminal deoxynucleotidyl transferase and dNTP having a mercapto group. In this reaction, the mercapto group was introduced in an end of the polynucleotide. The end-labeled polynucleotide was spotted on a gold electrode to allow the polynucleotide to chemically absorbed to the substrate. In this way, a gene-sensor type DNA chip was prepared.

As described above, according to the embodiment of the present invention, it is possible to specifically detect the endocrine disrupting action of a test substance. Furthermore, it is possible to easily detect a transcriptional product whose transcription amount varies depending upon an endocrine disrupting substance. Moreover, the detection of the transcriptional product makes it easier to detect the toxicity of a test substance, that is, the endocrine disrupting action. Therefore, the toxicity, i.e., endocrine disrupting action, of wasting materials and novel materials can be checked without fail.

### EXAMPLE 6

Convenient In-vitro Assay System for Exogenous Endocrine Disrupting Substance

1. Cultivation of Neuro2a

An anionic exchange resin AG1-X8 and fetal bovine serum were mixed at a ratio of 50 mg resin/mL and incubated at room temperature for 5 hours. After centrifugation at 1000×g for 10 minutes, the resin was removed. A fresh resin was added at a ratio of 50 mg resin/mL to the resultant mixture and subjected to a further incubation at room temperature for 18 hours. Thereafter, centrifugation was performed at 1000×g for 10 minutes and 30,000×g for 20 minutes to completely remove the resin. The resultant mixture was sterilized by a filter having pours of 0.22 μm diameter to prepare triiodothyronine-removed fatal bovine serum. The fatal bovine serum (10%) was mixed with DF medium containing Dulbecco's MEM medium and ham F-12 medium at a ratio of 1:1. In this medium, murine neuroblastoma, Neuro2a, were cultured, at 37° C. in a 5% CO₂.

The cultured cells were distributed into two vessels. Thirty nM triiodothyronine was added to one of the culture solutions and did not added to the other vessel. The two vessels were incubated for a further 5 days.

2. In Vitro Assay for Dioxin

To each of the two types of cells obtained in the step 1, 1 nM of an exogenous endocrine disrupting substance, TCDD (2378-tetrachlorodibenzo p-dioxin) was added and subjected to incubation for 24 hours. Thereafter, morphological changes of the cells were observed and the cells were recovered.

According to the following procedures, polynucleotides whose transcription amounts varied depending upon an exogenous endocrine disrupting substance except those whose transcription amounts varied by the toxicity inherent to the exogenous endocrine disrupting substance, were obtained.

3. Preparation of mRNA

To the cells (5×10⁶) obtained in the step 2, 18 ml of homogenate buffer (containing guanidineisocyanate, SDS, EDTA) was added and homogenized. After the homogenate was centrifuged at 15° C. at 5000×g for 20 minutes, the supernatant was taken. Subsequently, the supernatant was reciprocally moved 10 times through the injection needle of 18G connected to a syringe. The resultant solution was overlaid on cesium solution (containing CsTFA and EDTA) of the same amount and subjected to ultracentrifugation at 3000×g for 20 hours. After that, a centrifuge tube was taken out and the supernatant was gently sucked up by a pipette. The centrifuge tube was turned upside down on paper towel and allowed to dry. After the drying, the bottom of the centrifuge tube was cut off by a heated razor. The sediment (pellet) was washed with 70% ethanol and further dried. Thereafter, the pellet was suspended in sterile water to give a mRNA solution.

4. Subtraction of mRNA

Of mRNA solutions obtained from the two types of cells in Step 3 above, the RNA solution obtained from the cell incubated in the medium containing triiodothyronine was subjected to reverse transcription to synthesize cDNA. More specifically, cDNA was synthesized as follows: To the solution containing 1 μg of RNA, a reverse transcriptase buffer, dNTP mix (10 μM for each), an RNAase inhibitor (10 u), a random primer (25 pmol), and M-MLV reverse transcriptase (20 u) were added to make up 20 μL of the total volume. The solution was subjected to reactions performed at 30° C. for 10 minutes, 42° C. for 20 minutes, and 99° C. for 5 minutes, to synthesize cDNA. RNase was added in the cDNA solution to remove RNA. With this procedure, a single-stranded DNA solution was prepared. This solution and the mRNA solution obtained in Step 3 from the cells incubated in the medium without triiodothyronine, were combined to allow a hybridization reaction at 42° C. The hybridization reaction solution was overlaid on the cesium solution of the same amount, and subjected to ultracentrifugation at 30,000×g for 20 hours. A DNA solution containing an unhybridized single chain DNA was recovered to afford a desired polynucleotide solution.

### EXAMPLE 7

Identification of a Protein Having a Polysaccharide Chain Causing a Conformational Change Depending Upon an Endocrine Disrupting Substance.

1. Preparation of a Protein

To the cells (5×10⁶) obtained in Example 6, an immidazole buffer containing 1% Triton X-1000 was added in an amount 5 fold as high as the amount of the cells. The resultant was homoginazed. Thereafter, the resultant solution was allowed to stand alone at 4° C. overnight with stirring. The reaction solution was centrifuged at 1,000,000×g for 60 minutes. The supernatant solution was regarded as a crude protein extract solution.

2. Immobilization of Lectin to Sepharose

To 2 mL of coupling buffer (0.1M acetic buffer of pH6.4 containing 1M of CaCl₂, 1 mM of MnCl₂, 1 mM of MgCl₂, 0.2M methyl α-D mannoside), 50 mg of concanavalin A, and 12 mg of NaBH₃CN were dissolved. To the reaction mixture, 0.5% damp formyl Sepharose previously expanded with a coupling solution, was added and allowed to react at 4° C. overnight. Furthermore, to perform the cross-linkage of subunits, the gel obtained was reacted at 24° C. for 8 hours in a solution containing 48 mg of NaBH₃CN in 2 ml of an aqueous solution of 1% glutaraldehyde containing 0.2M methyl α-D-mannoside. Finally, to block the remaining formyl group, the gel was suspended in a 2 ml of 0.1M Tris buffer (pH7.4) containing 6.2 mg of NaBH₃CN and allowed to react at room temperature for one hour.

3. Treatment of Protein in a Lectin Column

To a protein extract solution extracted by the method described in Example 2, step 1 from the cells cultured according to the method described in Example 6, concanavalin A-Sepharose was added and soaked at 37° C. for 3 hours. Thereafter, the Sepharose was packed into a column and sufficiently washed with imidazole buffer containing Triton X-100. The elution of a protein from the column was performed by using 100 mM α-methylmannoside/1% Triton X-100/50 mM imidazole buffer.

4. Cleavage of a Polysaccharide Chain of a Protein

To the protein solution recovered from the column of the step 3, a glycopeptidase F was added and reacted at 37° C. for one hour. After the reaction, the reaction solution was heated up to 100° C. to inactivate an enzyme. Furthermore, the protein solution extracted in Step 1 mentioned above was treated with glycopeptidase F in the same condition as mentioned above.

5. Preparation of a Gel for Two-dimensional Electrophoresis

(1) Preparation of an Isoelectric Capillary Gel

A gel solution was prepared as follows. The composition of each of solution components is shown in a parenthesis. 0.2 mL of A solution (0.23% TEMED), 1.0 mL of B solution (16% acryl amide, 0.8% bis-acryl amide), 0.2 mL of C₁ solution (40% Ampholine, pH 3.5-10), 0.05 mL of C₂ solution (40% Ampholine, pH 3.5-10), 0.4 mL of S solution (5% Nonidet P-40), and 1.92 g of urea were mixed on ice. After individual components were dissolved, 0.2 mL of D solution (20% ammonium persulfate) was added to degas the solution. 3.5 mL of the gel solution obtained was added in a cylindrical container and a capillary is placed in the container. The remaining gel solution was added to the container by a syringe and allowed to stand alone for 30 minutes.

(2) Preparation of a Gel Containing SDS

A SDS-containing gel was prepared as follows. The composition of each of solution components is shown in a parenthesis. 4.5 mL of A solution (0.04N HCl, 18.2% Tris, 0.1% SDS), 6 mL of C solution (30% acryl amide, 0.3% Bis), and 7.5 mL of water were mixed. After degassed, 4.5 μL of TEMED and 54 μL of D solution (0.1% ammonium persulfate) were added to the resultant solution and mixed. 16 mL of the obtained solution was placed in a flat-plate gel forming mold. After 1 mL of distilled water was overlaid on the mold, the mold was allowed to stand at room temperature for about one hour to obtain 10% gel. 1.5 mL of B solution (0.04N HCl, 6% Tris, 0.01% SDS), 0.6 mL of C solution, and 3.9 mL of water were mixed. After degassed, 1.5 μL of TEMED and 18 μL of D solution were added and mixed to a concentrated gel solution was prepared. Water present on an electrophoretic gel was discarded and an upper surface of the gel was washed with a small amount of the concentrated gel solution. Thereafter, the mold was filled with 3.2 mL of the concentrated gel solution.

6. Two-dimensional Electrophoresis

The cathode vessel of an isoelectric electrophoretic apparatus was equipped with the isoelectric capillary gel prepared as mentioned above. Subsequently, a cathode vessel solution (0.04 M NaOH) was added and air bubbles were removed. Thereafter, four types of protein samples prepared in Step 4 were individually added to the capillaries. Electrophoresis was performed by supplying 0.1 mA constant current per capillary gel. When the voltage reaches 300V, a constant voltage was applied. Thereafter, electrophoresis was performed for 80 minutes. After completion of the electrophoresis, the isoelectric gel was removed and put into a gel mold filled with an equilibrium buffer (0.06N HCl, 15 μL/mL Tris, 2% SDS, 5% 2-mercaptoethanol, 0.002% BPB solution) to expose to the upper surface of a flat-plate gel. After allowed to stand alone for 15 minutes, the equilibrium buffer was removed. An electrode vessel solution (6 g/L Tris, 28.8 g/L glycine, 0.1% SDS, 0.0002% BPB solution) was added to upper and lower layers of a vessel for use in the second dimensional electrophoresis. Electrophoresis was performed while supplying a current as low as 10 mA per a single gel plate. The composition of each of solution components is shown in parenthesis.

7. Gel Staining

After completion of the two-dimensional electrophoresis performed in accordance with Step 6, the gel was removed from the glass plate and placed in a plastic container containing distilled water to wash the gel. After the washing, the distilled water in the container was discarded and a staining solution (having ethanol containing 0.25% Coomassie Brilliant Blue R-250:acetic acid:water at a ratio of 9:2:2) was added in a volume sufficient to cover the gel. The staining was performed for 2 hours while the staining solution was allowed to stand. The staining solution was removed.

Instead, a decolor solution 1 (a mixture of ethanol:acetic acid:water=25:8:65) was added. The decolor treatment was performed for 4 hours. Thereafter, the decolor solution 1 was removed. Subsequently, a decolor solution 2 (a mixture of ethanol:acetic acid:water=10:15:175) was added. Decolor treatment was performed until a gel portion containing no protein became transparent. The composition of each of the solution components is shown in parentheses.

8. Identification of a Protein Having a Polysaccharide Chain Causing a Conformational change Due to an Endocrine Disrupting Substance

Protein spots on the gel obtained in Step 7 were compared between a sample containing a test substance and a sample containing no test substance. The spots different in intensity were identified.

9. Excision of a Protein from the Gel

The protein identified in Step 8 was excised from the gel by a cutter knife. The gel piece was cut into small pieces of about 2 mm width and placed in a dialysis tube filled with 20 mM Tris buffer (pH8.0) containing 0.1% SDS. The dialysis tube was placed in a beaker containing about 1 L of SDS Tris buffer. Dialysis was performed overnight while stirring with a magnetic stirrer. The extracting solution was recovered on the following day. After confirming that the gel piece was not stained with a pigment, the extracting solution was passed through a filter to remove the gel pieces. The extracting solution was transferred to a centrifuge tube made of glass. Acetone previously cooled was added to the centrifuge tube in a volume 5 folds as large as that of the extracting solution, and allowed to stand still at −80° C. for 2 hours. After the stand-still, the resultant solution was centrifuged at 10,000 rpm for 30 minutes. A proteinaceous sediment precipitated by acetone was recovered. The sediment obtained was dried at room temperature and dissolved in 0.05 mL of 0.1% SDS.

10. Determination of an N-terminal Amino Acid sequence of the protein recovered

The protein obtained in Step 9 was subjected to amino acid sequence automatic analyzer to determine the N-terminal amino acid sequence.

11. Preparation of a Polyclonal Antibody Recognizing a Protein

A synthetic oligopeptide was prepared from the N-terminal amino acid sequence determined in Step 10. The synthetic oligopeptide, which would act as an immunogen, was injected into a rabbit abdominal cavity. In this manner, a polyclonal antibody was prepared.

12. Preparation of Antibody Affinity Column

Bromcyan-activated Sepharose 4B was soaked in water and allowed to expand well. After 2 mL of Sepharose 4B was washed with A solution (i.e., NaHCO₃ containing 0.5 M NaCl). Subsequently, 5 mg of the polyclonal antibody obtained in Step 3 dissolved in 2 ml of A solution was added to the mixture. The resultant solution was allowed to stand still at room temperature for 2 hours to immobilize the polyclonal antibody to the Sepharose.

13. Purification of a Protein

Sepharose having an antibody immobilized thereon was packed in a column. 50 mL of proteinaceous extract solution obtained in the same manner as in the method described in Example 2, Step 1 was diluted to 2-folds by a buffer solution containing 50 mM ammonium sulfate. The diluted solution was applied to the column equilibrated with buffer B at a flow rate of 5 mL/hour. After the column was washed with buffer A containing 0.1M ammonium sulfate, a protein bound to the column was extracted.

### EXAMPLE 8

Preparation of a Monoclonal Antibody Recognizing a Polysaccharide Chain Causing a Conformational Change Depending Upon the Action of an Endocrine Disrupting Substance

1. Preparation of Hybridoma

The protein isolated in Example 7 and a complete adjuvant were taken in an equal amount and loaded into 1 mL of a syringe. The syringes were connected to each other by a joint and stirred well until they were emulsified. The resultant emulsion was injected into a mouse abdominal cavity to immunize the mouse. At two weeks after a first injection for immunization, a second immunization was performed. Two week later, additional immunization was performed. On the third day after the additional immunization, cell fusion was performed as follows. The cervical vertebrae of the immunized mouse was dislocated, the spleen was enucleated and placed in a 6 cm-dish containing 5 mL of RPMI. Excessive fat was removed from the spleen and washed by using two new 6 cm-dishes containing 5 mL of RPMI. The washed spleen was dragged and transferred between half-folded stainless metal grids of a 5 cm square, and centrifugally washed twice with 10 mL of RPMI. To the precipitated cells, 0.17M of NH₄Cl was added and the cells were soaked in ice, thereby inducing hemolysis. After the hemolysis, 5 mL of RPMI was added to the sample and centrifuged at 1600 rpm for 5 minutes. The volume of the resultant solution was brought to 20 mL to obtain a spleen cell suspension. On the other hand, 2 to 4 dishes of myeloma cells in the logarithmic growth period were centrifuged at 1200 rpm for 5 minutes and recovered. The resultant myeloma cells were washed twice with serum-free RPMI, and suspended in 10 mL of RPMI to prepare a myeloma cell suspension.

The number of cells of each of the spleen cell suspension and myeloma cell suspension was counted. Based on the counts, the myeloma cell suspension was added to the spleen cell suspension such that the ratio of the myeloma cells to the spleen cells falls within the range of ⅕ to 1/20. This cell suspension was centrifuged for 5 minutes to remove the supernatant. To the precipitate obtained, 0.3 mL of 50% polyethylene glycohol 1500 was added in a stroke and immediately stirred well. While stirring, 40 mL of RPMI was further added to the precipitate and subjected to centrifugation at 1000 rpm for 5 minutes. To the precipitate obtained, 50 mL of HAT medium (i.e., 1×10⁻⁷M of hypoxanthine, 4×10⁻⁴M of aminopterin, PRMI-FCS containing 16×10⁻⁴M of thymidine in a final concentration) was added. The resultant solution was seeded 96-wells of each of three plates in an amount of 100 μL/well. After 4 and 5 days, fresh HAT medium was added to all wells in an amount of about 100 μL/well. As a result, proliferation of hybridoma cells were observed in almost all wells in about a week.

2. Purification of a Polysaccharide Chain from a Protein

To the protein obtained in Example 7, 1/100 of pronase P was added and allowed to react at 37° C. overnight, thereby decomposing the peptide chain moiety of the protein. After the decomposition, the reaction solution was subjected to the lectin column treatment in the same manner as described in Example 7, Step 3 by use of the lectin column prepared in Example 7, Step 2. In this manner, the polysaccharide chain of the protein was purified.

3. Preparation of an Assay Plate Having a Polysaccharide Chain Immobilized

To 2.5 mg of the polysaccharide chain purified in Step 2, 80 μL of water and 200 μL of pyrimidine were added and dissolved, and then, a pyrimidine solution containing 9.9 mg of anhydrous palmitate was immediately added to react them at 37° C. for 6 hours. After the reaction, pyrimidine and water were removed under a reduced pressure. Subsequently, unreacted anhydrous palmitate and a side product, palmitate, were extracted with ether to obtain an acylated polysaccharide chain. The acylated polysaccharide chain thus obtained was dissolved by adding 40 μL of water and 200 mL of pyrimidine and subjected again to an acylation reaction. The acylated polysaccharide chain was finally dissolved in a 98% aqueous pylimidine solution and stored. The acylated polysaccharide chain pyrimidine solution was diluted with water to make a polysaccharide chain solution having a concentration of 100 μg/mL. The polysaccharide chain solution was distributed by 20 μL to each of the wells of an assay plate and allowed to stand still at room temperature for one hour. Thereafter, the assay plate was placed in a desiccator containing phosphorus pentoxide. The desiccator was vacuumed by a water-stream aspirator to vaporize a solvent. In this way, the assay plate was prepared.

4. Selection of a Hybridoma Producing an Antibody Recognizing a Polysaccharide Chain

The assay plate prepared in Step 3 above was subjected to a blocking treatment by use of 1% BSA-PBS for one hour. To each of the wells, 20 μL of the 2-fold dilution of the supernatant of the hybridoma prepared in Step 1 was added and allowed to stand still at 4° C. for 16 hours. Subsequently, the wells were washed with 1% BSA-PBS three times to remove unbound antibodies. To each of the wells, 50 μL of a solution of protein A labeled with a fluorescence was added and allowed to stand still at room temperature for 2 hours. Thereafter, the wells were washed with 1% BSA-PBS to remove unbound protein A. The assay plate was observed by a fluorescent detector. A hybridoma present in a well was screened based on fluorescence emission. In the manner mentioned above, the hybridoma producing the antibody specifically recognizing the polysaccharide chain of a protein was screened.

5. Cloning

The hybridoma cells were collected from the wells emitting fluorescence in the aforementioned Step 4. An aliquot was taken form the hybridoma cells and the number of the cells was counted by a hemocytometer. Based on the count, the hybridoma cells were diluted into an appropriate concentration. After that, 40 hybridoma cells were mixed with the spleen cells of about 1×10⁸ prepared from a mouse in 40 mL of RPMI. The cell suspension mixture was distributed to 96-wells of each of two plates by 200 μL per well and incubated at 37° C. The incubation was continued for about 2 weeks while replacing the medium with a fresh medium twice in a week. After two weeks, the supernatant was taken from each of wells. After the activity of the antibody was checked, the hybridomas showing the antibody activity were collected. The hybridomas thus collected was incubated by a 24-well plate. When the density of the hybridoma cells increased sufficiently high, the cells were transferred to a 35 mm plate and incubation was further continued. The same operation was repeated once more. The hybridomas thus established through the steps were stored in a refrigerator.

6. Screening of a Monoclonal Antibody Recognizing a Polysaccharide chain

0.5 mL of pristine was injected to the abdominal cavity of about 4-week old mouse. About one week after the pristine injection, 500 mL of the suspension solution of the hybridoma cells obtained in Step 5, which were dispersed in RPMI at a concentration of about 4×10⁶ cell/mL, was injected in the abdominal cavity. The hybridoma cells used herein were prepared by thawing the hybridoma cells stored in a refrigerator, incubating and dispersing them in RPMI at a concentration of about 4×10⁶ cells/mL. About two weeks later, the ascites was accumulated in the abdominal cavity of the mouse and expand the abdomen. At that stage, the abdomen of the mouse was cut to collect the ascites by a Pasteur's pipette. The ascites was added to a 15 mL centrifuge tube containing 0.2 mL of NaN₃EDTA solution, and centrifuged at 2000 rpm for 10 minutes. The resultant supernatant was regarded as a monoclonal antibody solution.

### EXAMPLE 9

Detection of a Polysaccharide Chain Causing a Conformational Change Depending Upon an Endocrine Disrupting Action

A protein was extracted, in the same manner as described in Example 7, Step 1, from the cells exposed to an endocrine disrupting substance in accordance with the method of Example 1. The protein extracted was subjected to two-dimensional electrophoresis in the same manner as described in Example 7, Step 6. After completion of the electrophoresis, the gel was removed form a glass substrate and placed in a plastic container having distilled water therein. After the gel was washed, proteins contained in the gel were transferred onto a PVDF film by an electro-transfer method. After the transfer, the PVDF film was soaked in a Blockase (trade name, manufactured by Snow Brand Milk Products Co., Ltd.) to perform a blocking reaction at room temperature for 2 hours. Subsequently, the resultant PVDG film was soaked in the monoclonal antibody diluted with PBS obtained in Example 7 to react with the monoclonal antibody at room temperature for 2 hours. After 2 hours, the PVDF film was transferred to TBS and washed with TBS three times at room temperature for 15 minutes. The PVDF film was soaked in 10% blockace containing an anti-mouse rabbit secondary antibody labeled with peroxidase and allowed to react at room temperature for one hour. After completion of the reaction with the secondary antibody, the PVDF film was soaked in a coloring solution (containing 50 mg/mL of DAB, 50 μM Tris HCl buffer (pH7.5), peroxidase) to allow the film to produce color. Based on the observation as to whether a spot develops a color or not, the presence or absence of the endocrine disrupting action of a test substance is detected.

### EXAMPLE 10

Preparation of Protein Chip (1)

The protein chip of the present invention was prepared as follows. A glass substrate coated with biotinylated BSA was further coated with streptoavidin. The antibody recognizing a polysaccharide chain obtained in Example 7 was biotinylated and immobilized onto the glass substrate by a commercially available spotter. In this manner, a fluorescence detection type protein chip was prepared. The protein chip made it possible to detect the endocrine disrupting action of a test substance based on a conformational change of a polysaccharide chain.

### EXAMPLE 11

Preparation of a Protein Chip (2)

The antibody recognizing a polysaccharide chain obtained in Example 8 was spotted on a gold electrode by a commercially available spotter to allow the antibody to chemically bind to the gold electrode. In this manner, a current-detection type protein chip was prepared. The protein chip thus prepared made it possible to detect the endocrine disrupting action of a test substance based on a conformational change of a polysaccharide chain.

As described in the foregoing, according to an embodiment of the present invention, it is possible to produce a novel detection method for detecting an endocrine disrupting action based on the conformational change of a polysaccharide chain.

Hence, if the test is performed by using the cell, such as Neuro2a, having a sensitivity to an endocrine hormone as a detection cell in the presence of the endocrine hormone such as triiodothyronine, it is possible to specifically detect the toxicity of the endocrine disrupting substance.

Additional advantages and modifications will readily occur to those skilled in the art. therefore, the invention in its broader aspects is not limited to the specific details and representative embodiments shown and described herein. accordingly, various modifications may be made without departing from the spirit or scope of the general inventive concept as defined by the appended claims and their equivalents.

What is claimed is:

1. A method of detecting an endocrine disrupting action of a test substance, comprising:
(1) culturing a cell having a sensitivity to an endocrine hormone in a first culture system in which endocrine hormone and the test substance are present;
(2) determining the presence or absence of an endocrine disrupting action of the test substance by comparing a first gene expression pattern obtained from the cell of the first culture system with a second gene expression pattern expressed by a cell having a sensitivity to the endocrine hormone in the presence of the endocrine hormone, thereby detecting a gene specific to the first gene expression pattern.

2. A method of detecting an endocrine disrupting action of a test substance, comprising:
(1) (a) culturing a cell having a sensitivity to an endocrine hormone in a first culture system in which an endocrine hormone and the test substance are present;
(b) culturing the cell having a sensitivity to the endocrine hormone in a third culture system in which the endocrine hormone is absent and the test substance is present; and
(2) determining the presence or absence of an endocrine disrupting action of the test substance by obtaining a first gene expression pattern obtained from the cell in the first culture system and a third gene expression pattern obtained from the cell in the third culture system, comparing the first gene expression pattern with the third gene expression pattern and a second expression pattern expressed by a cell having a sensitivity to the endocrine hormone in the presence of the endocrine hormone, thereby detecting a gene specific to the first gene expression pattern.

3. A method of detecting an endocrine disrupting action of a test substance, comprising:
(1) (a) culturing a cell having a sensitivity to an endocrine hormone in a first culture system in which the endocrine hormone and the test substance are present;
(b) culturing the cell a having a sensitivity to the endocrine hormone in a third culture system in which the endocrine hormone is absent and the test substance is present;
(2) determining the presence or absence of an endocrine disrupting action of the test substance by comparing a first gene expression pattern obtained from the cell of the first culture system with a third gene expression pattern obtained from the cell in the third culture system, thereby detecting a gene specific to the first gene expression pattern.

4. A method of detecting an endocrine disrupting action of a test substance, comprising:
(1) (a) culturing a cell having a sensitivity to an endocrine hormone in a first culture system in which the endocrine hormone and the test substance are present;
(b) culturing the cell a having a sensitivity to the endocrine hormone in a second culture system in which the endocrine hormone is present and the test substance is absent; and
(c) culturing the cell a having a sensitivity to the endocrine hormone in a third culture system in which the endocrine hormone is absent and the test substance is present; and
(2) determining the presence or absence of an endocrine disrupting action of the test substance by comparing a first gene expression pattern obtained from the cell in the first culture system with a second gene expression pattern obtained from the cell in the second culture system and the third gene expression pattern obtained from the cell in the third culture system, thereby detecting a gene specific to the first gene expression pattern.

5. The method according to any one of claims 1 to 4, wherein said endocrine hormone is selected from the group consisting of estrogen, estradiol, progesterone, androgen, testosterone, androsterone, cortisol, aldosterone, corticosterone, cortison, triiodothyronine, and tyroxicin; and said cell having a sensitivity to the endocrine disrupting action is selected from the group consisting of Neuro2a, S-20Y , MCF7, TM3, TM4 and 15P-1.

6. The method according to any one of claims 1 to 4, wherein said endocrine hormone is triiodothyronine and said cell having a sensitivity to the endocrine hormone is Neuro2a.

7. The method according to any one of claims 1 to 4, wherein comparison of the gene expression patterns are made by comparing bands obtained by subjecting a gene group contained in each of gene expression patterns to electrophoretic separation.

8. The method of detecting an endocrine disrupting action of a test substance according to any one of claims 1 to 4, comprising
(a) recovering RNAs from each of the culture systems of (1);
(b) subjecting the RNAs recovered in the step (a) to reverse transcription;
(c) amplifying reverse transcription products obtained in (b) by PCR; and
(d) subjecting PCR products obtained in the step (c) to electrophoresis, comparing electrophoretic patterns of bands obtained, thereby detecting a band specific to a first gene expression pattern.

9. The method according to any one of claims 1 to 4, wherein said gene expression patterns are compared by hybridizing gene groups contained in each of the gene expression patterns with each other, and subtracting unhybridized genes.

10. A method of detecting an endocrine disrupting action of a test substance, comprising:
(1) determining the presence or absence of an endocrine disrupting action of the test substance by
(a) culturing a cell having a sensitivity to an endocrine hormone in a first culture system in which the endocrine hormone and the test substance are present;
(b) culturing a cell having a sensitivity to the endocrine hormone in a second culture system in which an endocrine hormone is present and test substance is absent;
(c) culturing a cell having a sensitivity to the endocrine hormone in a third culture system in which the endocrine hormone is absent and the test substance is present; and
(d) culturing a cell having a sensitivity to the endocrine hormone in a fourth culture system in which both the endocrine hormone and the test substance are absent;
(2) (a) isolating RNA from the first culture system and preparing a first cDNA based on the RNA;
(b) isolating a second RNA from the second culture system;
(c) isolating a third RNA from the third culture system; and
(d) isolating RNA from the fourth culture system and preparing a fourth cDNA based on the RNA;
(3) (a) hybridizing the first cDNA and the second RNA and recovering unhybridized cDNA;
(b) hybridizing the third RNA and the fourth cDNA, recovering unhybridized RNA; and
(4) hybridizing the cDNA obtained in (a) of (3) and the RNA obtained in (b) of (3); and recovering unhybridized RNA, thereby detecting a gene specific to an endocrine disrupting action.

11. A polynucleotide obtained in a method comprising the following, and a complementary polynucleotide having a complementary sequence of said polynucleotide,
(1) (a) culturing a cell having a sensitivity to an endocrine hormone in a first culture system in which the endocrine hormone and a test substance are present;
(b) culturing a cell having a sensitivity to the endocrine hormone in a second culture system in which the endocrine hormone is present and the test substance is absent; and
(c) culturing a cell having a sensitivity to the endocrine hormone in a third culture system in which the endocrine hormone is absent and the test substance is present; and
(2) obtaining first, second, third gene expression patterns from the first, second, third culture systems, and comparing the first, second, third gene expression patterns with each other, thereby detecting and isolating a gene specific to the first gene expression pattern.

12. The polynucleotide and a complementary polynucleotide having a complementary sequence to said polynucleotide, according to claim 11,
wherein said endocrine hormone is selected from the group consisting of estrogen, estradiol, progesterone, androgen, testosterone, androsterone, cortisol, aldosterone, corticosterone, cortison, triiodothyronine, and tyroxicin; and said cell having a sensitivity to the endocrine disrupting action is selected from the group consisting of Neuro2a, S-20Y , MCF7, TM3, TM4 and 15P-1.

13. A DNA chip comprising the polynucleotide and complementary polynucleotide according to claim 11 as a nucleic acid probe.

14. A method of detecting an endocrine disrupting action of a test substance, comprising:
(1) culturing a cell having a sensitivity to an endocrine hormone in a first culture system with the endocrine hormone and the test substance; and
(2) determining the presence or absence of an endocrine disrupting action of the test substance by comparing a first glycoprotein pattern obtained from the cell of the first culture system with a second glycoprotein pattern expressed by a cell having a sensitivity to the endocrine hormone in the presence of the endocrine hormone, thereby detecting a glycoprotein specific to the first glycoprotein pattern.

15. A method of detecting an endocrine disrupting action of a test substance, comprising:
(1) (a) culturing a cell having a sensitivity to an endocrine hormone in a first culture system with the endocrine hormone and the test substance; and
(b) culturing a cell having a sensitivity to the endocrine hormone in a third culture system in which the endocrine hormone is absent and the test substance is present; and
(2) determining the presence or absence of endocrine disrupting action of the test substance by
obtaining a first glycoprotein pattern obtained from the cell of the first culture system and a third glycoprotein pattern obtained form the cell of the third culture system, and
comparing the first glycoprotein pattern with the third glycoprotein pattern and a second glycoprotein pattern obtained from a cell having a sensitivity to the endocrine hormone in the presence of the endocrine hormone, thereby detecting a glycoprotein specific to the first glycoprotein pattern.

16. A method of detecting an endocrine disrupting action of a test substance, comprising:
(1) (a) culturing a cell having a sensitivity to an endocrine hormone in a first culture system in which the endocrine hormone and the test substance are present; and
(b) culturing a cell having a sensitivity to the endocrine hormone in a third culture system in which the endocrine hormone is absent and the test substance is present; and
(2) determining the presence or absence of endocrine disrupting action of the test substance by
comparing the first glycoprotein pattern obtained from the cell of the first culture system with the third glycoprotein pattern obtained from the cell of the third culture system, thereby detecting a glycoprotein specific to the first glycoprotein pattern.

17. A method of detecting an endocrine disrupting action of a test substance, comprising:
(1) (a) culturing a cell having a sensitivity to an endocrine hormone in a first culture system in which the endocrine hormone and the test substance are present;
(b) culturing a cell having a sensitivity to the endocrine hormone in a second culture system in which the endocrine hormone is present and the test substance is absent; and
(c) culturing a cell having a sensitivity to the endocrine hormone in a third culture system in which the endocrine hormone is absent and the test substance is present; and
(2) determining the presence or absence of an endocrine disrupting action of the test substance by comparing first, second and third glycoprotein patterns obtained respectively from the cells of the first, second, third culture systems, thereby detecting a glycoprotein specific to the first glycoprotein pattern.

18. The method according to any one of claims 14 to 17, wherein said endocrine hormone is selected from the group consisting of estrogen, estradiol, progesterone, androgen, testosterone, androsterone, cortisol, aldosterone, corticosterone, cortison, triiodothyronine, and tyroxicin; and said cell having a sensitivity to the endocrine disrupting action is selected from the group consisting of Neuro2a, S-20Y , MCF7, TM3, TM4 and 15P-1.

19. The method according to any one of claims 14 to 17, wherein said endocrine hormone is triiodethyronine and said cell having a sensitivity to the endocrine hormone is Neuro2a.

20. The method according to any one of claims 14 to 17, wherein the glycoprotein specific to the first culture system is performed by a method comprising:
(a) extracting proteins biosynthesized by a cell contained in each of culture systems of (1);
(b) recovering a glycoprotein by binding a substance capable of specifically binding to a polysaccharide chain contained in the proteins extracted in (a) to the polysaccharide chain;
(c) recovering a protein contained in the glycoprotein by cutting off the polysaccharide chain; subjecting the protein to electrophoresis, thereby obtaining a glycoprotein pattern for each of the culture systems; and
(d) comparing glycoprotein patterns to each other.

21. An antibody against a substance susceptible to an endocrine disrupting action, said antibody being prepared by a method comprising:
(1) (a) culturing a cell having a sensitivity to an endocrine hormone in a first culture system in which the endocrine hormone and a test substance are present;
(b) culturing a cell having a sensitivity to the endocrine hormone in a second culture system in which the endocrine hormone is present and the test substance is absent; and
(c) culturing a cell having a sensitivity to the endocrine hormone in a third culture system in which the endocrine hormone is absent and the test substance is present; and
(2) extracting proteins biosynthesized by a cell contained in each of the first, second and third culture systems of (1);
(3) recovering a glycoprotein by binding a substance capable of specifically binding to a polysaccharide chain contained in the proteins extracted in (2) to the polysaccharide chain;
(4) recovering a protein contained in the glycoprotein by cutting off the polysaccharide chain; subjecting the protein to electrophoresis, thereby obtaining a glycoprotein pattern for each of the first, second and third culture systems;
(5) comparing the glycoprotein patterns to each other, thereby detecting a glycoprotein specific to the first culture system, and further recovering a protein contained in the glycoprotein;
(6) preparing a polyclonal antibody against the protein obtained in (5),
(7) recovering a desired glycoprotein from the proteins extracted in (2) by the polyclonal antibody obtained; and
(8) preparing an antibody against to a polysaccharide chain contained in the glycoprotein obtained in (7).

22. The antibody against the polysaccharide chain susceptible to an endocrine disrupting action, according to claim 21, wherein said endocrine hormone is selected from the group consisting of estrogen, estradiol, progesterone, androgen, testosterone, androsterone, cortisol, aldosterone, corticosterone, cortison, triiodothyronine, and tyroxicin; and said cell having a sensitivity to the endocrine disrupting action is selected from the group consisting of Neuro2a, S-20Y , MCF7, TM3, TM4 and 15P-1.

23. A protein chip comprising the antibody according to claim 21.
